(19) United States
(12) Patent Application Publication
(10) Pub. No.: US 2026/0354854 A1
(43) Pub. Date: Nov. 12, 2026

(54) INSTRUMENT AND TECHNIQUE FOR RELEASING SOFT TISSUE AT JOINTS IN THE FOOT

(71) Applicant: Treace Medical Concepts, Inc., Ponte Vedra, FL (US)

(72) Inventors: Paul Dayton, Ankeny, IA (US); Daniel J. Hatch, Greeley, CO (US); Robert D. Santrock, Morgantown, WV (US); W. Bret Smith, Lexington, SC (US); William T. DeCarbo, Cranberry Township, PA (US); Mark E. Easley, Durham, NC (US); Jody McAleer, Jefferson City, MO (US); Jared White, St. Augustine, FL (US); Whitney Moore, St Johns, FL (US)

(21) Appl. No.: 19/629,126

(22) Filed: Mar. 26, 2026

Related U.S. Application Data
(60) Provisional application No. 63/778,346, filed on Mar. 26, 2025.

(51) Int. Cl. A61B 17/32

(52) U.S. Cl. CPC A61B 17/32

(57) ABSTRACT

A method of releasing soft tissue at a metatarsophalangeal joint may involve surgically accessing a metatarsophalangeal joint between a first metatarsal and a proximal phalanx. The method can also involve moving a head of an instrument about a distal end of the first metatarsal. The instrument can include a concave inner surface, a convex outer surface opposite the concave inner surface, and a sharpened leading edge where the concave inner surface transitions to the convex outer surface. Moving the head of the instrument about the distal end of the first metatarsal may involve positioning the concave inner surface facing the distal end of the first metatarsal and using the sharpened leading edge to cut soft tissue on a medial side, a lateral side, and a plantar side of the distal end of the first metatarsal.

### CROSS-REFERENCE

This application claims the benefit of and priority to United States Provisional Patent Application No. 63/778,346, filed Mar. 26, 2025, the entire contents of which incorporated herein by reference.

### TECHNICAL FIELD

This disclosure relates to instruments and techniques for performing a bone realignment and/or fusion procedure and, more particularly, to instruments and techniques for releasing soft tissue during the procedure.

### BACKGROUND

The human foot includes the five toes (which are also known as the “phalanges”) and their connecting long bones (or “metatarsals”). The joint between a metatarsal and a phalange is called the metatarsophalangeal (“MTP”) joint. Several small bones together comprise a phalanx or toe. Four of the five toes have three phalanx bones respectively connected by two joints. The big toe (or “hallux”) has two phalanx bones distal and proximal with a joint in between called the interphalangeal joint. The big toe articulates with the head of the first metatarsal at the first metatarsophalangeal joint (the “MTP” joint) and there are two tiny, round bones called sesamoids on the plantar side of the metatarsal head. The phalanges are connected to the metatarsals at the ball of the foot. The two sesamoid bones are located underneath the first metatarsal bone and assist in flexion of the big toe at the first MTP joint.

Hallux rigidus is a degenerative disease of the first metatarsophalangeal joint. It is the most common form of arthritis in the foot, reportedly affecting 1 in 40 people over the age of 50. The first MTP joint plays an important functional role during the gait cycle as it carries a significant amount of an individual's body weight with each step. Osteophyte formation and degeneration of the cartilage occurs dorsally in early stages of the disease and progresses to involve the entire first MTP joint. Consequently, individuals with hallux rigidus experience joint pain and decreased range of motion in the sagittal plane. This leads to altered gait mechanics and significant reduction in activity and quality of life for patients.

In some cases, surgical intervention is needed to address hallux rigidus and/or other bone issues. Surgical intervention may involve realigning and/or fusing one or more bones of the foot, improving patient comfort. A clinician may use a variety of different surgical instruments during a surgical procedure performed on the foot, including one or more cutting instruments to cut tissue and/or bone. Surgical instruments that can facilitate efficient, accurate, and reproducible clinical results are useful for practitioners performing bone realignment techniques.

### SUMMARY

In general, this disclosure is directed to orthopedic instruments and associated systems and techniques utilizing such instruments. In some examples, an orthopedic instrument according to the disclosure can be used to cut and release soft tissue, such as ligaments, at or adjacent the distal head of the first metatarsal. This can free the distal head of the first metatarsal from the opposed proximal phalanx at the metatarsophalangeal (“MTP”) joint for bone end preparation, realignment, and/or fusion. In some examples, the orthopedic instrument is additionally or alternatively used as a retractor to gain access to the distal head of the metatarsal for bone end preparation and/or as a shield during bone preparation.

In some implementations, the instrument is configured with a handle graspable by a clinician performing the procedure and a head operatively connected to the handle. The head can have a scooped body forming a concave inner surface and a convex outer surface separated by a thickness of the material defining the head. The head can have a sharpened leading edge where the concave inner surface transitions to the concave outer surface. The leading edge may be sharpened to come to an apex or point configured to cut tissue, as opposed to being a rounded or blunt edge. In either case, the clinician can surgically access the distal end of the first metatarsal at the metatarsophalangeal joint and use the instrument to release soft tissue. For example, the clinician can move the head in a sweeping motion causing the sharpened leading edge to advance along the medial side, lateral side, and/or plantar side of the distal end of the head to cut soft tissue and release the distal end of the metatarsal.

In some examples, the instrument is additionally or alternatively used as a shield to protect the proximal end of the proximal phalanx while preparing the distal end of the first metatarsal. For example, the concave inner face of the instrument may be positioned adjacent to and/or in contact with the proximal end of the proximal phalanx, presenting a convex outer face of the instrument facing the distal end of the first metatarsal. A reamer can then be applied to the distal end of the first metatarsal with the instrument shielding inadvertent contact between the reamer and the proximal end of the proximal phalanx.

While the disclosed instruments and associated techniques can be useful at the first metatarsophalangeal joint, the instruments and/or techniques may also be used at other joints of the foot. For example, instruments and/or techniques according to the disclosure may be adapted for use in joints in which soft tissue release, joint mobilization, bone preparation, tissue protection, and/or controlled access to a bone surface is desired. In some configurations, an instrument may be configured to engage and/or be positioned adjacent a selected bone surface and be advanced relative to surrounding tissue to release capsular tissue, ligamentous tissue, fibrous tissue, tendon-associated tissue, and/or other soft tissue structures that restrict motion or access within a joint. The instrument may additionally or alternatively be used to retract tissue, protect adjacent structures, facilitate joint distraction, and/or shield a bone surface during preparation or correction procedures.

As one example, the disclosed instruments and techniques may be used at the talonavicular joint between the talus and the navicular. For example, the instrument may be introduced adjacent the joint and manipulated to release soft tissue along one or more aspects of the joint in order to improve joint mobility, facilitate repositioning, and/or support preparation of one or both joint surfaces for corrective treatment, realignment, arthrodesis, or other intervention.

As another example, the disclosed instruments and techniques may be used at the subtalar joint between the calcaneus and the talus. For example, the instrument may be positioned within or adjacent the joint space and moved relative to surrounding tissue to release soft tissue that limits repositioning of the hindfoot. The instrument may also be used to assist with joint mobilization, tissue protection, and preparation of opposed bone surfaces in connection with deformity correction, arthrodesis, and/or other procedures involving the subtalar joint.

As a further example, the disclosed instruments and techniques may be used at one or more lesser metatarsophalangeal joints. For example, the instrument may be dimensioned and shaped for use at the second, third, fourth, and/or fifth metatarsophalangeal joints to release soft tissue, improve joint mobility, facilitate reduction or realignment, and/or support preparation of bone surfaces in connection with corrective procedures, fusion procedures, or other treatments.

In still further examples, the disclosed instruments and techniques may further be used at an interphalangeal joint of a lesser toe, such as a proximal interphalangeal joint, including in connection with a hammer toe release procedure. For example, the instrument may be used to release contracted soft tissue associated with the joint, facilitate joint distraction and exposure, protect adjacent structures, and/or improve access for bone resection, contouring, implant placement, fixation, or other corrective treatment.

For example, in a hammer toe correction procedure involving a proximal interphalangeal joint of a lesser toe, the disclosed instrument may be used to release soft tissue associated with the joint and to facilitate exposure of the opposed bone ends that define the joint. After soft tissue release, one or both of the bone ends may be prepared by resection, contouring, reshaping, planing, and/or other joint preparation to remove tissue, create complementary surfaces, and/or facilitate correction of the deformity. The phalanx may then be repositioned relative to an adjacent phalanx to reduce flexion deformity, restore a more anatomically desired alignment, and/or place the toe in a corrected orientation. In some applications, fixation may then be applied to maintain the corrected position during healing, such as by using a pin, wire, screw, implant, staple, and/or other fixation construct extending across or otherwise associated with the proximal interphalangeal joint. Accordingly, the disclosed instruments and techniques may be used not only for soft tissue release, but also to support joint preparation, phalangeal realignment, and fixation in connection with hammer toe correction.

Accordingly, in various applications, instruments and techniques disclosed for use at the first metatarsophalangeal joint may also be adapted for use at multiple forefoot, midfoot, and hindfoot joints. An instrument according to the disclosure may be adapted for use at a particular forefoot, midfoot, and/or hindfoot joint by modifying one or more dimensional, geometric, structural, and/or functional characteristics of the instrument to correspond to the anatomy and procedural requirements of a selected joint. For example, the instrument may be configured in different sizes, lengths, widths, thicknesses, curvatures, profiles, and/or working-end configurations to accommodate differences in joint space, bone contour, depth of access, surrounding soft tissue, and/or desired range of motion or exposure. In some implementations, the instrument may additionally or alternatively be modified with respect to angulation, rigidity, flexibility, edge configuration, surface contour, handle configuration, and/or material construction so as to improve access, control, tissue release, retraction, shielding, distraction, and/or bone preparation at a particular target location. A disclosed instrument may be configured for use at a single specific joint or may be implemented to be suitable for use across different joints of the foot while preserving one or more underlying functional principles of the disclosure.

In one example, a method of releasing soft tissue at a metatarsophalangeal joint is described. The method involves surgically accessing a metatarsophalangeal joint between a first metatarsal and a proximal phalanx and moving a head of an instrument about a distal end of the first metatarsal. The example specifies that the instrument includes a concave inner surface, a convex outer surface opposite the concave inner surface, and a sharpened leading edge where the concave inner surface transitions to the convex outer surface. The example also specifies that moving the head of the instrument about the distal end of the first metatarsal involves positioning the concave inner surface facing the distal end of the first metatarsal and using the sharpened leading edge to cut soft tissue on a medial side, a lateral side, and a plantar side of the distal end of the first metatarsal.

In another example, a method of releasing soft tissue at a joint of a foot is described. The method includes surgically accessing a joint between a first bone and a second bone and moving a head of an instrument relative to the first bone and/or the second bone at the joint. The instrument may include a concave inner surface, a convex outer surface opposite the concave inner surface, and a sharpened leading edge at a transition between the concave inner surface and the convex outer surface. The method can involve moving the head of the instrument by positioning the concave inner surface facing an end of the first bone or an end of the second bone and using the sharpened leading edge to cut soft tissue adjacent the joint so as to mobilize the first bone relative to the second bone.

In another example, an instrument configured for cutting soft tissue at an end of a first bone or an end of a second bone defining a joint in a foot is described. The instrument includes a handle and a head coupled to the handle. The head may include a concave inner surface extending from a first widthwise side edge of the head to a second widthwise side edge of the head and along at least a portion of a length of the head, and a convex outer surface opposite the concave inner surface. In some examples, the concave inner surface transitions to the convex outer surface along a leading edge, and the leading edge is sharpened along at least a portion of its length. The head may be sized to be inserted around the end of the first bone and/or around the end of the second bone and configured to cut soft tissue for releasing the joint.

In another example, an instrument configured for cutting soft tissue at a distal head of a first metatarsal in a foot is described. The instrument includes a handle extending from a first end to a second end and a head on the second end of the handle. The head incudes a concave inner surface extending from a first widthwise side edge of the head to a second widthwise side edge of the head and along at least a portion of a length of the head. The head also includes a convex outer surface opposite the concave inner surface. The example specifies that the concave inner surface transitions to the convex outer surface along a leading edge, and the leading edge is sharpened along at least a portion of a length of the leading edge. The example also states that the head is sized to be inserted around a distal end of a first metatarsal with the concave inner surface facing the distal end of the first metatarsal, including plantarly of the distal end of a first metatarsal and in an intermetatarsal space between the first metatarsal and a second metatarsal of a foot.

The details of one or more examples are set forth in the accompanying drawings and the description below. Other features, objects, and advantages will be apparent from the description and drawings, and from the claims.

### BRIEF DESCRIPTION OF DRAWINGS

FIG. 1A-1D are different views of an example orthopedic instrument according to the disclosure

FIGS. 2A and 2B are top and front views, respectively, of a foot showing example metatarsal alignment positions.

FIG. 3 illustrates the different anatomical planes of the foot.

FIG. 4 is a superolateral view of the bone-ligament-capsular anatomy of the first MTP joint.

FIGS. 5-8 illustrates an example procedure steps that can be performed during a surgical technique utilizing an instrument according to the disclosure.

FIGS. 9A-9D are different views of another example configuration of the orthopedic instrument according to the disclosure.

FIG. 10 is a perspective view of an example portion of a foot showing a lesser metatarsophalangeal joint between a lesser proximal phalanx and a lesser metatarsal being released using an instrument according to the disclosure.

FIG. 11 is a perspective view of an example portion of a foot showing a proximal interphalangeal joint between a middle phalanx and a proximal phalanx being released using an instrument according to the disclosure.

FIG. 12 is a perspective view of an example portion of a foot showing a talonavicular joint between a navicular and a talus being released using an instrument according to the disclosure.

FIG. 13 is a perspective view of an example portion of a foot showing a subtalar joint between a talus and a calcaneus being released using an instrument according to the disclosure.

### DETAILED DESCRIPTION

This disclosure generally relates orthopedic instruments and associated systems (e.g., kits) and techniques incorporating one or more such instruments. In some examples, an instrument according to the disclosure can be used cut one or more ligaments and/or other soft tissue for mobilizing a bone for subsequent preparation and/or realignment. For example, the instrument may be used to perform cut and release soft tissue during a proximal phalanx realignment procedure. In these applications, the instrument can be used to cut one or more ligaments and/or other soft tissue adjacent a distal end of the metatarsal opposite the proximal end of the proximal phalanx. For example, the instrument can be inserted through an incision accessing an intermetatarsal space between a first metatarsal and an adjacent proximal phalanx. The instrument may have a concave surface that can be placed against the generally convex distal end of the first metatarsal. A sharpened leading edge of the instrument can then be used to cut one or more ligaments and/or other targeted tissue. This can mobilize the first metatarsal. While the instrument may find particular utility at the metatarsophalangeal joint, in practice, the clinician can use the instrument to cut any targeted soft tissue, including for releasing other bones and/or joints in the foot, without departing from the scope of the disclosure.

In addition to or in lieu of use at the metatarsophalangeal joint, the disclosed device and associated techniques may be used at other joints, particularly joints formed by relatively small bones, such as joints in the foot. For example, the device may be used at forefoot, midfoot, and/or hindfoot joint defined by a first bone and a second bone, and in some applications may also be used at joints in other parts of the anatomy having comparable access constraints and soft tissue structures. In such applications, the device may be selected, sized, shaped, and/or otherwise configured based on the anatomy of the target joint and the desired surgical objective while preserving the ability of the device to access a confined joint region and engage tissue adjacent the first bone and/or the second bone. Accordingly, the disclosed device need not be limited to a particular metatarsophalangeal application, but instead may be adapted for use at a variety of joints where controlled soft tissue release and bone access are beneficial.

In some examples, the device may be introduced into or adjacent a joint between the first bone and the second bone and manipulated to cut, separate, release, and/or otherwise disrupt soft tissue that restricts motion, exposure, or repositioning of the joint. For example, the device may be used to release capsular tissue, ligamentous tissue, fibrous tissue, tendon-associated tissue, and/or other targeted tissue in order to mobilize the first bone relative to the second bone for realignment, deformity correction, reduction, and/or other repositioning. In some configurations, the head of the device may have a scooped or otherwise concave shape configured to wrap partially or fully around an end of a bone. Such a configuration may facilitate controlled advancement of the head along the bone end while maintaining the head adjacent the bone surface, thereby improving release of soft tissue attached to, surrounding, or otherwise adjacent the bone end.

In exemplary applications, the devices, systems, and techniques can be used during a surgical procedure performed on one or more bones, such as a bone alignment, osteotomy, fusion procedure, fracture repair, and/or other procedures where one or more bones are to be set in a desired position. Such a procedure can be performed, for example, on bones (e.g., adjacent bones separated by a joint or different portions of a single bone) in the foot or hand, where bones are relatively small compared to bones in other parts of the human anatomy. In one example, a procedure utilizing devices and/or techniques of the disclosure can be performed by preparing the metatarsophalangeal (“MTP”) joint for fusion, realigning the phalanx relative to the metatarsal, and/or applying fixation across the MTP to hold the prepared end of the phalanx relative to the prepared end of the metatarsal for fusion.

Preparation and fusion of a metatarsal and phalanx may be performed according to the disclosure for a variety of clinical reasons and indications. Preparation and fusion of a metatarsal and proximal phalanx at the MTP joint may be performed to treat hallux rigidus, hallux valgus, and/or other bone and/or joint conditions.

Hallux rigidus is characterized as a degenerative arthritis of the MTP joint, particularly the first MTP joint. The cause of hallux rigidus is often unclear. While arthritis can be caused by traumatic or iatrogenic injuries that directly cause damage to the articular cartilage of the MTP joint, most commonly the aetiology of hallux rigidus is idiopathic. Patients may have family history and/or bilateral involvement leading to hallux rigidus. As hallux rigidus progresses, the normal coupling of the center of rotation of the proximal phalanx and metatarsal head may be disrupted, leading to eccentric gliding of proximal phalanx on the metatarsal head. Osteophytes may form preferentially on the dorsal surface. Further, while a normal first MTP motion may have approximately 75° of dorsiflexion and 35° of plantarflexion, patients with hallux rigidus may typically exhibit a decreased range of motion, such as dorsiflexion less than 60°, such as less than 40°, less than 30°, or even less than 20°.

Hallux valgus, also referred to as hallux abducto valgus, is a complex progressive condition that is characterized by lateral deviation (valgus, abduction) of the hallux and medial deviation of the first metatarsophalangeal joint. Hallux valgus typically results in a progressive increase in the hallux abductus angle, the angle between the long axes of the first metatarsal and proximal phalanx in the transverse plane. An increase in the hallux abductus angle may tend to laterally displace the plantar aponeurosis and tendons of the intrinsic and extrinsic muscles that cross over the first metatarsophalangeal joint from the metatarsal to the hallux. Consequently, the sesamoid bones may also be displaced, e.g., laterally relative to the first metatarsophalangeal joint, resulting in subluxation of the joints between the sesamoid bones and the head of the first metatarsal. This can increase the pressure between the medial sesamoid and the crista of the first metatarsal head.

While systems, techniques, and devices are described herein particularly in connection with the first metatarsal and first proximal phalanx of the foot, the techniques and devices may be used on other bone ends and/or adjacent bone pairs separated by a joint. For example, the techniques and devices may be performed on a different metatarsal (e.g., second, third, fourth, or fifth metatarsal) and its opposed proximal phalanx or on a metatarsal and its opposed cuneiform at the tarsometatarsal joint.

The techniques and devices may be used at other adjacent bone pairs in the foot, including bone pairs at forefoot, midfoot, and/or hindfoot joints. For example, the disclosed cutting instrument may be used between adjacent phalanges of a lesser toe, such as between a proximal phalanx and a middle phalanx at a proximal interphalangeal joint, or between a middle phalanx and a distal phalanx at a distal interphalangeal joint. In other examples, the disclosed cutting instrument may be used between a talus and a navicular, between a calcaneus and a talus, between a calcaneus and a cuboid, between a navicular and a cuneiform, between adjacent cuneiforms, and/or between a cuneiform and an adjacent metatarsal. In these and other examples, the dimensions, curvature, and overall profile of the head may be selected based on the size and contour of the bone end to be engaged while preserving the ability of the head to be advanced along the bone end and to release soft tissue adjacent the joint.

Independent of the specific joint at which the instrument is used, the disclosed cutting instrument may be used to release capsular tissue, ligamentous tissue, fibrous tissue, and/or other soft tissue spanning or surrounding a selected joint in order to mobilize a first bone relative to a second bone. Such mobilization may facilitate realignment, reduction, deformity correction, bone preparation, fixation, arthrodesis preparation, and/or other treatment steps involving the adjacent bone pair. For example, after soft tissue release, one or both bone ends defining the joint may be resected, cut, contoured, or otherwise prepared, and the first bone and/or second bone may be repositioned (e.g., moving the first bone relative to the second bone) and fixated relative to each other using a pin, screw, staple, implant, and/or other fixation construct. Accordingly, the disclosed techniques and devices may be adapted for use across different joints and bone pairs while employing the same general principle of advancing a cutting head along a bone end to release soft tissue adjacent the joint.

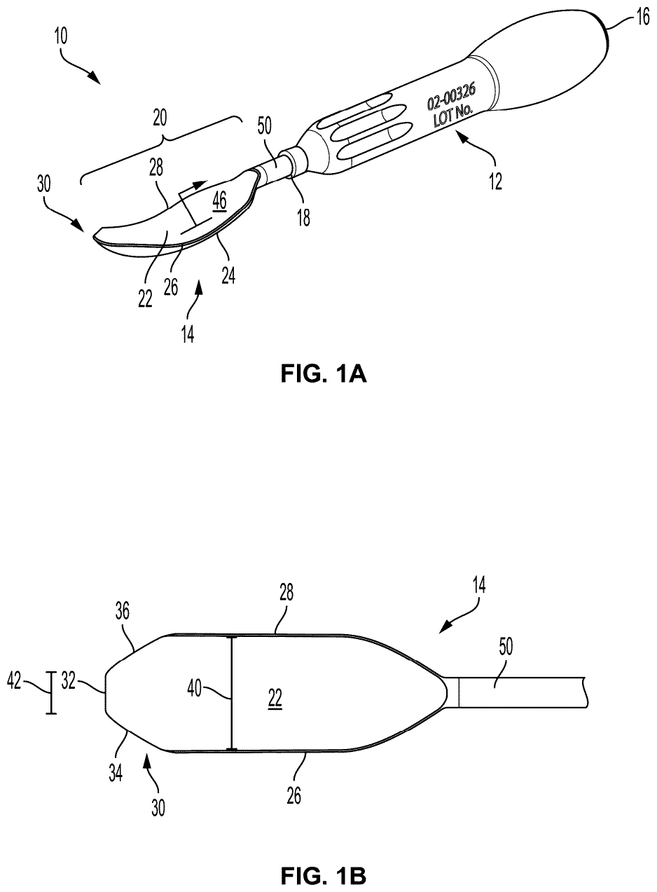

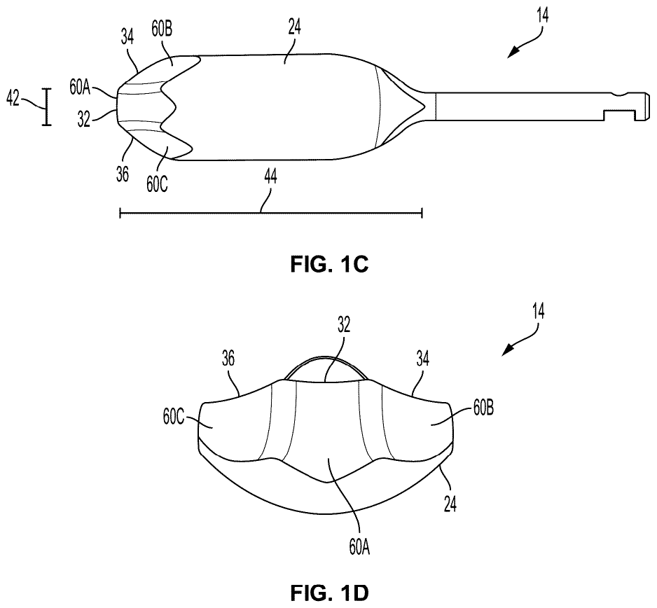

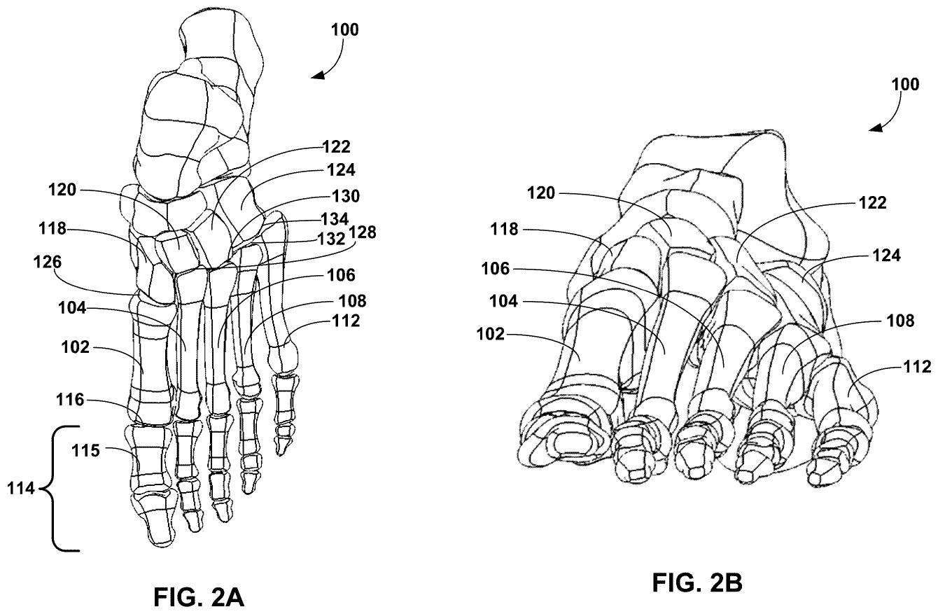

The anatomy of the foot and example techniques utilizing a cutting instrument according to the disclosure will be described in greater detail with respect to FIGS. 2-9. However, an example orthopedic instrument according to the disclosure will first be described with respect to FIGS. 1A-1D.

FIGS. 1A-1D (collectively “FIG. 1”) are different views of an example orthopedic instrument 10 according to the disclosure. FIG. 1A is a top perspective view of instrument 10. FIG. 1B is an enlarged top view of an example configuration of the head of the instrument. FIG. 1C as a bottom view of an example configuration of the head of the instrument, shown with handle removed for purposes of illustration. FIG. 1D is an enlarged front view of the head of the instrument.

In the illustrated example of FIG. 1, instrument 10 includes a handle 12 and a head 14 operatively connected to the handle. For example, in the illustrated configuration, handle 12 has a major length extending from a first end 16 to a second end 18. Head 14 is connected to and extends from the second end 18 of handle 12. In various implementations, handle 12 and head 14 may be fabricated as a unitary body (e.g., cut or cast as a single piece of material) or may be fabricated as separate components that are joined together to form a combined structure the does not separate during use.

As shown, head 14 can define a scooped body region 20 formed by a concave inner surface 22 on one side and a convex outer surface 24 on an opposite side, separated by a thickness of material. For example, the scooped body region 20 of head 14 can have a concave inner surface 22 extending from a first widthwise side edge 26 to a second widthwise side edge 28. Head 14 can extend to a distal or leading edge 30 where the concave inner surface 22 transitions to the convex outer surface 24. The leading edge 30 can be sharpened to present a sharp leading face of head 14 that can be advanced against and cut through soft tissue during use in an orthopedic procedure.

In use, a clinician can advance a head 14 of instrument 10 toward target tissue to be cut, such as one or more ligaments to be cut. Head 14 of instrument 10 can be configured (e.g., sized and/or shaped) to be inserted and moved around the end of a bone to release soft tissue around the end of the bone, such as the distal end of a metatarsal. For example, the concave inner surface 22 of head 10 may be sized and shaped relative to the size and shape of the end of the bone against which the concave inner surface may be positioned adjacent. In some examples, concave inner surface 22 of head 10 is sized relative to the distal end of a first metatarsal, such that the distal end of the first metatarsal can be partially or fully received within the concavity defined by concave inner surface 22.

In either case, the clinician can use instrument 10 to release soft tissue during a procedure. The clinician may make an incision to access the metatarsophalangeal joint separating the first metatarsal from a proximal phalanx. The clinician may use a scalpel to cut soft tissue on the medial and/or lateral sides of the metatarsophalangeal joint and/or the distal end of the first metatarsal. In some examples, the clinician may also use a scalpel to cut the joint capsule of the metatarsophalangeal joint between the distal end of the first metatarsal and the proximal end of the proximal phalanx to allow the metatarsal to be separated from the proximal phalanx at the joint.

The clinician may use the straight scalpel to cut soft tissue parallel to and along the medial side of the distal end of the first metatarsal and parallel to and along the lateral side of distal end of the first metatarsal. The clinician can further use instrument 10 to cut and release other soft tissue not cut and released by the scalpel. For example, the clinician may move head 14 of instrument 10 relative to the distal end of the first metatarsal and use the sharpened leading edge 30 to cut soft tissue on the medial side of the distal end of the first metatarsal, on the lateral side of the distal end of the first metatarsal, and/or on the plantar side of the distal end of the first metatarsal. The clinician may place concave inner surface 22 of head 14 facing and optionally in contact with the distal end of the first metatarsal (which may generally be rounded and/or convex in shape).

The clinician can advance head 14 of instrument 10 in a distal to proximal direction toward the proximal end of the first metatarsal and/or in a sweeping or arc motion (e.g., from the medial side of the bone to the plantar side of the bone to the lateral side of the bone, or vice versa) to cut tissue about the distal end of the first metatarsal. Instrument 10 can cut and release any desired soft tissue during use. Example soft tissue that may be targeted and cut by head 14 of instrument 10 can include, but is not limited to, a medial collateral ligament, a lateral collateral ligament, and/or one or more sesamoid suspensory ligaments. The configuration of instrument 10 and head 14 can facilitate efficient and thorough release of soft tissue adjacent the distal end of the bone.

While instrument 10 can be used for any desired surgical procedure, in some configurations, the cutting instrument is configured (e.g., sized and shaped) to facilitate cutting within a joint space between opposed bones of the foot. For example, head 14 of instrument 10 may be configured to be inserted into at the MTP joint between a first metatarsal and an adjacent proximal phalanx. The clinician may insert head 14 of instrument 10 into the target joint space to cut soft tissue (e.g., muscles, tendons, ligaments, and/or facia) within the joint space. Such soft tissue may be connectively attached to one or more bones (e.g., metatarsal, sesamoid) intended to be realigned. Cutting the soft tissue using instrument 10 can mobilize the bone for subsequent realignment. For example, cutting the soft tissue using instrument 10 can mobilize the bone for subsequent preparation, realignment, and/or fusion. After preparation and other associated surgical steps, a realigned bone may be permanently fixated in a moved position using a fixation device to promote fusion of the realigned bone.

Head 14 can have a variety of different configurations. With reference to FIGS. 1B and 1D, head 14 is illustrated as including a leading cutting surface 32, a first side cutting surface 34, and a second side cutting surface 36. Leading cutting surface 32 may define the distal-most cutting surface of head 14 (with the first end 16 of handle 12 defining the proximal-most portion of instrument 10). First side cutting surface 34 and second side cutting surface 36 can be separated from each other in the widthwise direction across head 14, e.g., where the width of the head is perpendicular to the length of instrument 10 and the material thickness of the head. First side cutting surface 34 can extend angularly outwardly from leading cutting surface 32 to first widthwise side 26 of the head. Second side cutting surface 36 can extend angularly outwardly from leading cutting surface 32 in a direction opposite first side cutting surface 34 to second widthwise side 26 of head 14. As a result, the overall width of head 14 in the configuration of FIG. 1 enlarges moving from leading cutting surface 32 of the head proximally along the portion of the head defined by first and second side cutting surfaces 34, 36.

Configuring head 14 with a comparatively narrow leading cutting surface 32 trailed by a comparatively wider region of the cutting head defined by the first and second side cutting surfaces 34, 36 can be useful to facilitate controlled penetration of the head by the clinician during use. The comparatively narrow leading cutting surface 32 allows the clinician to apply concentrated force to a comparatively small leading cutting surface for introducing the instrument into a joint space and/or applying cutting force to tissue to be cut. The comparatively wider trailing region of head 14 expands out the region of cut created by the head. This can increase the cutting surface area of head 14, increasing the amount of tissue cut while at the same time providing increased resistance against the cutting head. This can help prevent the clinician from inadvertently plunging head 14 too deep into a joint space during the procedure, which can otherwise cut muscle, tendons, and/or other tissue not desired to be cut during the procedure.

In some implementations, cutting head 14 defines a maximum width 40 at a location along the head between first widthwise side edge 26 and second widthwise side edge 28. This maximum width 40 of head 14 can be compared to a length 42 of leading cutting surface 32, e.g., the linear cutting surface extending widthwise at the distal tip of head 14. The length 42 of leading cutting surface 32 can be set less than the maximum width 40 of head 14, thereby configuring head 14 with a comparatively narrow distal cutting tip that expands widthwise in a proximal direction.

While the specific dimensions of head 14 can vary depending on the intended application, in some examples, leading cutting surface 32 defines a length 42 ranging from 2 mm to 10 mm, such as from 4 mm to 8 mm, or from 5 mm to 7 mm. For example, leading cutting surface 32 may define a length 42 of approximately 6 mm (e.g., ±10 percent). In addition, the maximum width 40 of head 14 between first and second widthwise side edges 26, 28 may range from 10 mm to 40 mm, such as from 15 mm to 20 mm, or from 16 mm to 18 mm. For example, the maximum width 40 of head 14 may be approximately 17 mm (e.g., ±10 percent). Depending on the relative widthwise dimensions of head 14, a ratio of the length 42 of the leading cutting surface 32 divided by the maximum width 40 of the head may be less than 0.5, such as less than 0.45, or less than 0.4.

In some implementations, the maximum width 40 of head 14 may be selected based on the anatomy of the target joint and the intended procedure. For example, for a comparatively smaller joint application, such as release of a proximal interphalangeal joint during a hammer toe procedure, head 14 may define a maximum width 40 of about 6 mm, such as within a range from 4 mm to 8 mm, or from 5 mm to 7 mm. In another example, for a lesser metatarsophalangeal joint release procedure, head 14 may define a maximum width 40 of about 11 mm, such as within a range from 8 mm to 14 mm, or from 9 mm to 13 mm. These comparatively smaller width configurations may facilitate insertion into smaller joint spaces while still providing sufficient lateral span to engage and release soft tissue adjacent the joint.

A comparatively larger width configuration of head 14 may be used for larger joints and/or fusion procedures. For example, for a subtalar joint fusion procedure and/or a talonavicular joint fusion procedure, head 14 may define a maximum width 40 of about 21 mm, such as within a range from 16 mm to 26 mm, or from 18 mm to 24 mm. The comparatively greater width may provide increased coverage across the joint surfaces, increased engagement with tissue adjacent the joint, and/or increased resistance to unintended over-advancement of the head during the release procedure. Accordingly, the maximum width 40 of head 14 may be scaled for use across different foot joints, including lesser toe joints, lesser metatarsophalangeal joints, and hindfoot and/or midfoot joints.

In some configurations, cutting head 14 defines an overall length over the portion of head 14 defining concave inner surface 22 and convex outer surface 24 within a range from 25 mm to 100 mm, such as from 30 mm to 80 mm, or from 40 mm to 60 mm (e.g., 50 mm±10%) Handle 12 can have any desired length between first end 16 and second end 18 although, in some examples, may exhibit a length ranging from 50 mm to 200 mm, such as from 100 mm to 150 mm.

In some configurations, the length of head 14 over the portion of head 14 defining concave inner surface 22 and convex outer surface 24 may be selected based on the anatomy of the target joint and the intended procedure. For example, for a comparatively smaller joint application, such as release of a proximal interphalangeal joint during a hammer toe procedure, head 14 may define a length of about 9 mm, such as within a range from 6 mm to 12 mm, or from 7 mm to 11 mm. In another example, for a lesser metatarsophalangeal joint release procedure, head 14 may define a length of about 15 mm, such as within a range from 11 mm to 19 mm, or from 13 mm to 17 mm. These comparatively shorter length configurations may facilitate insertion and manipulation within smaller joint spaces while still providing sufficient surface extent to engage and release soft tissue adjacent the joint.

A comparatively greater length configuration of head 14 over the portion of head 14 defining concave inner surface 22 and convex outer surface 24 may be used for larger joints and/or fusion procedures. For example, for a subtalar joint fusion procedure and/or a talonavicular joint fusion procedure, may be greater than 25 mm, such as greater than 30 mm, greater than 35 mm, greater than 40 mm, or greater than 50 mm. For example, the length may be within a range from 25 mm to 75 mm, such as from 30 mm to 60 mm. The comparatively greater length may facilitate engagement along a larger portion of the joint, greater wrapping and/or scooping around a bone end or joint surface, and/or improved release of tissue across a wider procedural area. Accordingly, the length of head 14 over concave inner surface 20 may be scaled for use across different foot joints, including lesser toe joints, lesser metatarsophalangeal joints, and hindfoot and/or midfoot joints.

The depth and radius curvature of the concavity defined by concave inner surface 22 can vary but may be sized relative to the intended application. The depth of the concavity defined by concave inner surface 22 may be measured from the trough or bottom most point of concave inner surface 22 to a peak or topmost perimeter point bounding the concave surface. In some examples, the depth 46 is within a range from 3 mm to 25 mm, such as 5 mm to 10 mm, or from 6 mm to 10 mm.

The radius of curvature for a concave surface can be considered the radius of a sphere that most closely approximates the curvature of the surface at a point. For a concave surface, which curves inward, this radius can be considered the distance from the center of curvature (the center of the sphere) to the point on the surface, where the center of curvature lies along the normal (perpendicular) to the surface at the point of measurement. The concavity defined by concave inner surface 22 may have a constant radius of curvature across the width and/or length of concave inner surface 22 or may vary across the width and/or length of the surface. In some configurations, concave inner surface 22 defines a radius of curvature within a range from 5 mm to 25 mm, such as from 10 mm to 20 mm, or from 12 mm to 16 mm.

First side cutting surface 34 and/or second side cutting surface 36 may extend angularly outwardly from leading cutting surface 32 along the entire length of head 14 or along only a portion of the length. For example, first side cutting surface 34 and/or second side cutting surface 36 may extend angularly outwardly from leading cutting surface 32 along a portion of the length of head 14 before changing direction to extend parallel to the length and/or back towards a longitudinal center of the cutting head. In these latter configurations, head 14 may define a comparatively narrow distal region, a comparatively narrow proximal region, and a comparatively wider intermediate region between the distal and proximal regions. In the configuration of FIG. 1, head 14 narrows from a region of maximum width 40 down to a shank 50 positioned proximally of scooped body region 20. Handle 12 can be attached to head 14 at shank 50.

Leading edge 30 of head 14 (encompassing leading cutting surface 32, first side cutting surface 34, and second side cutting surface 36 in the example of FIG. 1) can be sharpened. A sharpened edge can refer to the finely honed or refined edge that has been intentionally shaped to be thin and capable of easily cutting through materials. In some applications, the edge is sharpened through a process involving grinding or honing the edge to a fine point, allowing the edge to be sharp to slice soft tissue with comparatively minimal effort.

To help leading edge 30 of head 14 advanced through and cut soft tissue, convex outer surface 24 of head 14 may include one or cutting faces. With reference to FIG. 1C, for example, head 14 may include one or more cutting faces 60 extending from leading edge 30 proximally along at least a portion of a length of convex outer surface 24. In the illustrated arrangement, head 14 includes three cutting faces 60 positioned side-by-side from the first widthwise side edge to the second widthwise side edge of head 14, although the head can be configured with a smaller number of cutting faces (e.g., one, two) or larger number of cutting faces (e.g., four, five, or more) in different configurations.

In the illustrated arrangement, head 14 includes a leading cutting face 60A, a first side cutting face 60B, and a second side cutting face 60C. First side cutting face 60B and second side cutting face 60C can be separated from each other in the widthwise direction across head 14 by leading cutting face 60A.

In some configurations, handle 12 and head 14 are formed as a unitary structure (e.g., via casting, milling) defined by a single type of material. In other configurations, handle 12 and head 14 may be formed as separate structures joined together to couple the cutting head to the handle for subsequent use. In some such configurations, handle 12 may be formed of a different material (e.g., a polymeric material) then head 14 (which may be formed of metal material), e.g., for increased grip ability and/or comfort and holding. For example, handle 12 may define a receiving cavity at second end 18, and an end of cutting head 14 opposite leading cutting surface 32 can be inserted into the receiving cavity to interconnect the handle to the head. Fixation means (e.g., adhesive, screws, bolts, welding) may be used to permanently affix the head to the handle. In other configurations, head 14 may be detachably attached to handle 12 to allow the handle to be used with different interchangeable heads (e.g., each having the same configuration or having different configurations from each other).

Handle 12 may generally be configured to be gripped manually by the hand of a clinician using instrument 10. Handle 12 may have an enlarged cross-sectional size (e.g., width, thickness) relative to head 14 to provide a larger surface for grasping. In some configurations, handle 12 includes surface texturing, such as knobs, ribs, knurls, and/or other features that facilitate gripping of the handle without slippage. While handle 12 may generally be designed to be gripped manually by the hand of the clinician, in other configurations, handle 12 may be designed to be inserted into a powered hand instrument that can drive movement of head 14 via the handle.

As briefly discussed above, instrument 10 can be used during a variety of different procedures, including as part of a bone alignment procedure. In some examples, instrument 10 is utilized during a procedure in which one or more bones of the foot are realigned. To further understand example applications of use, the anatomy of the foot will be described with respect to FIGS. 2A and 2B.

FIGS. 2A and 2B are top and front views, respectively, of a foot 100 showing normal metatarsal alignment positions. Foot 100 is composed of multiple bones including a first metatarsal 102, a second metatarsal 104, a third metatarsal 106, a fourth metatarsal 108, and a fifth metatarsal 112. First metatarsal 102 is on a medial-most side of the foot while fifth metatarsal 112 is on a lateral-most side of the foot. The metatarsals are connected distally to phalanges 114 and, more particularly, each to a respective proximal phalanx. The joint 116 between a metatarsal and a corresponding opposed proximal phalanx is referred to as a metatarsophalangeal (“MTP”) joint. For example, first metatarsal 102 is connected to the first proximal phalanx 115 at the first MTP joint, labeled as joint 116 in FIG. 2A, although second, third, fourth, and fifth MTP joints are also illustrated in series adjacent to the first MTP joint.

The first metatarsal 102 is connected proximally to a medial cuneiform 118, while the second metatarsal 104 is connected proximally to an intermediate cuneiform 120, and the third metatarsal 106 is connected proximally to lateral cuneiform 122. The fourth and fifth metatarsals 108, 112 are connected proximally to the cuboid bone 124. The joint between a metatarsal and opposed bone (cuneiform, cuboid) is referred to as the tarsometatarsal (“TMT”) joint. FIG. 2A designates a first TMT joint 126, a second TMT joint 128, a third TMT joint 130, a fourth TMT joint 132, and a fifth TMT joint 134. The angle between adjacent metatarsals is referred to as the intermetatarsal angle (“IMA”).

In the example of FIGS. 2A and 2B, foot 100 is illustrated as having generally normally aligned metatarsals. Normal metatarsal alignment may be characterized, among other attributes, by a low intermetatarsal angle (e.g., 9 degrees or less, such as 5 degrees or less) between the first metatarsal and the second metatarsal. In addition, the lesser metatarsals may be generally parallel to a longitudinal axis bisecting the foot proximally to distally.

Standard medical planes of reference and descriptive terminology are employed in this disclosure. A sagittal plane divides a body into right and left portions. A coronal or frontal plane divides a body into anterior and posterior portions. A transverse plane divides a body into superior and inferior portions. Anterior means toward the front of a body. Posterior means toward the back of a body. Superior or cephalad means toward the head. Inferior or caudal means toward the feet or tail. Medial means toward the midline of a body (e.g., toward a plane of bilateral symmetry of the body). Lateral means away from the midline of a body or away from a plane of bilateral symmetry of the body. Proximal means toward the trunk of the body. Distal means away from the trunk. Dorsal means toward the top of the foot or other body structure. Plantar means toward the sole of the foot or toward the bottom of the body structure.

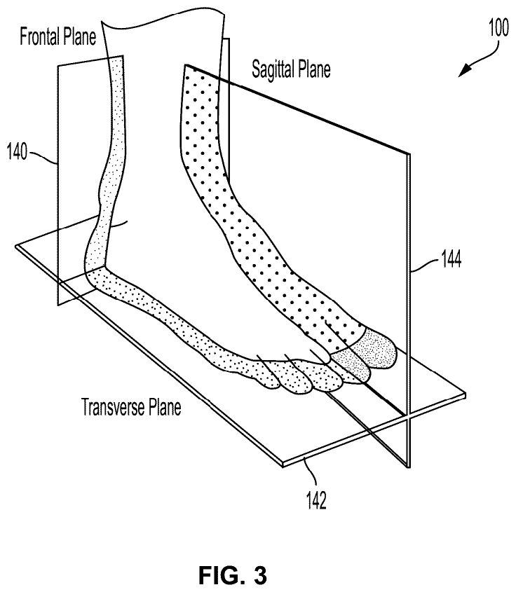

FIG. 3 illustrates the different anatomical planes of foot 100, including frontal plane 140, transverse plane 142, and sagittal plane 144. The frontal plane 140, which is also known as the coronal plane, is generally considered any vertical plane that divides the body into anterior and posterior sections. On foot 100, the frontal plane 140 is a plane that extends vertically and is perpendicular to an axis extending proximally to distally along the length of the foot. The transverse plane 142, which is also known as the horizontal plane, axial plane, or transaxial plane, is considered any plane that divides the body into superior and inferior parts. On foot 100, the transverse plane 142 is a plane that extends horizontally and is perpendicular to an axis extending dorsally to plantarly (top to bottom) across the foot. Further, the sagittal plane 144 is a plane parallel to the sagittal suture which divides the body into right and left halves. On foot 100, the sagittal plane 144 is a plane that extends vertically and intersects an axis extending proximally to distally along the length of the foot. For patients afflicted with certain bone misalignments, one or more of metatarsals may be deviated medially in the transverse plane (e.g., in addition to or in lieu of being rotated in the frontal plane and/or being deviated in the sagittal plane relative to clinically defined normal anatomical alignment for a standard patient population).

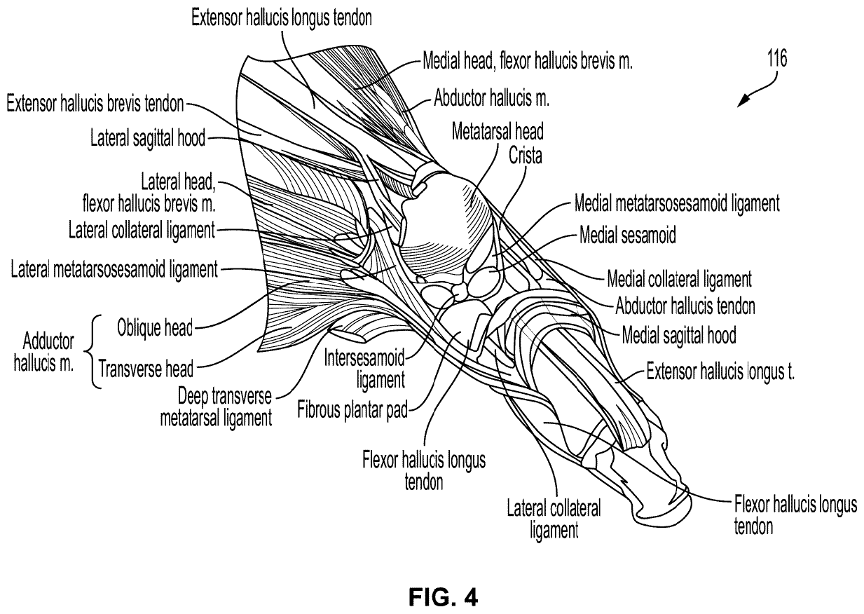

FIG. 4 is a superolateral view of the bone-ligament-capsular anatomy of the first MTP joint 116. As seen in FIG. 4, the first MTP joint 116 includes the distal end of the first metatarsal, the proximal end of the proximal phalanx, and the medial and lateral sesamoids. The sesamoid complex may be characterized as including two sesamoid bones, eight ligaments, and seven muscles. The sesamoid articulation with the first metatarsal head is in continuity with that of MTP articulation. Most of the capsuloligamentous stabilizing structures are concentrated on the plantar surface of the joint.

The dorsal surface of the MTP joint is dominated by the extensor hallucis longus tendon (EHL), which lies in the dorsal midline. The extensor hallucis brevis (EHB) tendon is just plantar and lateral to the EHL. The sagittal hood spreads out from the tendon sheath, encasing the ligaments to form a confluence of thickened capsular tissue extending plantarward to the collateral ligaments in the midline both medially and laterally. These capsuloligamentous structures generally align at the equator of the joint, extending from the upper condylar region of the first metatarsal head to the base of the proximal phalanx.

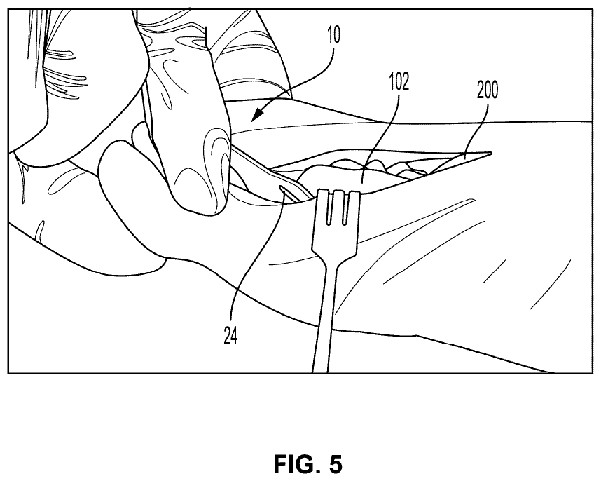

FIG. 5-8 illustrates an example procedure steps that can be performed during a surgical technique utilizing an instrument 10 according to the disclosure. With reference to FIG. 5, a technique may involve surgically accessing a metatarsophalangeal joint by making the incision 200 through the skin of the patient. In different applications, the incision may be a dorsal incision, a dorsal medial incision, or a medial incision. The incision may extend from 2 cm to 10 cm, such as from 4 cm to 8 cm over the metatarsophalangeal joint. The clinician can use a scalpel blade to create subcutaneous exposure of the distal end of first metatarsal 102 and the proximal end of proximal phalanx 115 across joint 116. The clinician can release the joint capsule with a scalpel to allow access to the joint. The clinician can then insert instrument 10 into the joint (e.g., into the joint capsule through the scalpel incision made into the joint capsule).

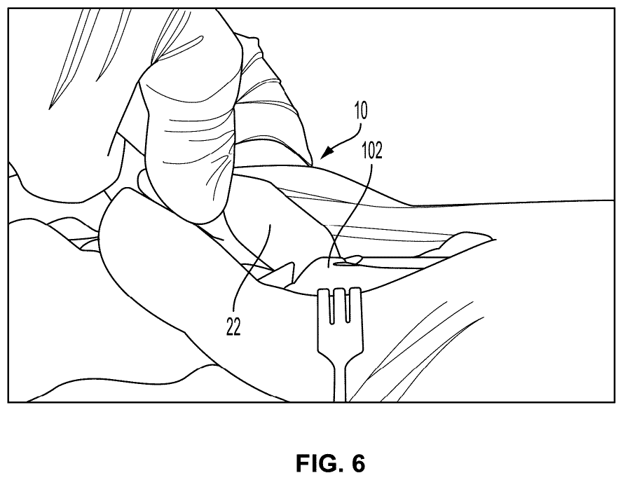

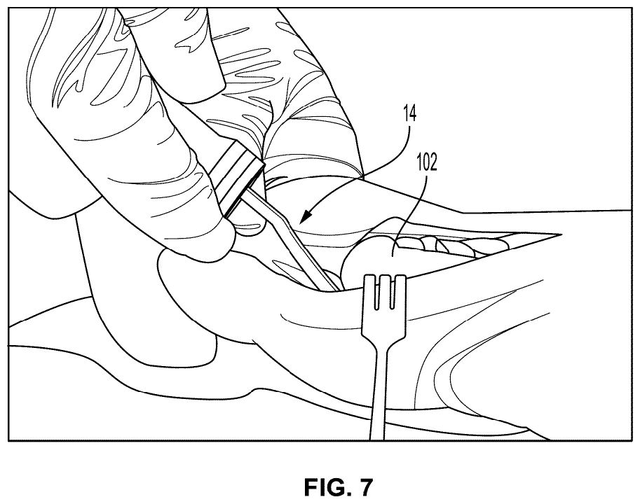

The clinician can advance the sharpened leading edge 30 of the instrument and a proximal to distal, distal to proximal reciprocating motion, and/or in a medial to lateral or lateral to medial motion. The clinician can move head 14 of instrument 10 on the medial and lateral aspects of the distal end of metatarsal 102. For example, FIG. 5 illustrates instrument 10 positioned on a medial side of the distal end of first metatarsal 102. FIG. 6 illustrates instrument 10 positioned on a lateral side of the distal end of first metatarsal 102. The clinician can also advance head 14 of instrument 10 under the plantar surface of the distal end of first metatarsal 102, as illustrated in FIG. 7. The clinician can position concave inner surface 22 of instrument 10 facing the metatarsal bone and the convex outer surface 24 of the instrument facing away from the bone as the clinician manipulates the instrument relative to the end of the bone.

In the process of moving instrument 10 relative to the distal end of the bone, the clinician can cut and release various soft tissue restraining the distal end of first metatarsal 102. For example, the clinician may cut in a release a medial collateral ligament, a lateral collateral ligament, and/or sesamoids to suspensory ligaments. It should be appreciated that the clinician need not move head 14 of instrument 10 in any particular order, and the clinician can engage the head of the instrument on the medial side, the lateral side, and/or the plantar side of the end of the bone in any particular order.

The clinician can additionally or alternatively release the proximal end of the proximal phalanx 115. For example, the clinician can position concave inner surface 22 of instrument 10 facing the proximal end of proximal phalanx 115 and move the instrument (e.g., in a distal to proximal direction) about the medial side, lateral side, and/or plantar side of the end of the bone to help mobilize the end of the bone.

Independent of whether or how the clinician mobilizes the distal end of first metatarsal 102 and/or the proximal end of proximal phalanx 115, the clinician can prepare the end of each bone forming the joint so as to promote fusion of the bone ends across the MTP joint following the procedure. Bone preparation may involve using a tissue removing instrument to apply a force to the end face of the bone so as to create a bleeding bone face to promote subsequent fusion. Example tissue removing instruments that can be used include, but are not limited to, a saw, a rotary bur, a rongeur, a reamer, an osteotome, a curette, and the like.

In some examples, the clinician prepares the distal end of first metatarsal 102 using a generally concave reamer (e.g., a cone reamer). The reamer can be advanced axially down a guide pin inserted into the face of the bone and rotated using a rotary instrument (e.g., drill). The reamer (or other instrument, if a different surgical instrument is used) may denude the distal end face of the first metatarsal 102 of remaining cartilage down to the subchondral bone plate. In some examples, the distal end face of the first metatarsal may be fenestrated in addition to or in lieu of one or more other end preparation steps, such as reaming the face with a reamer.

The proximal end face of proximal phalanx 115 may be prepared using the same preparation technique or techniques performed on first metatarsal 102 or using a different preparation technique or techniques. In instances where the end face of the first metatarsal is prepared using a generally conically-shaped reamer, the proximal end face of the proximal phalanx may be prepared using a corresponding generally convex reamer (e.g., a cup reamer). A guide pin may be inserted into the end face of the proximal phalanx and the convex reamer advanced down the guide pin and rotated using a rotary instrument. The reamer (or other instrument, if a different surgical instrument is used) may denude the end face of the proximal phalanx of remaining cartilage down to the subchondral bone plate. In some examples, the end face of the proximal phalanx may be fenestrated in addition to or in lieu of one or more other end preparation steps, such as reaming the face with the reamer.

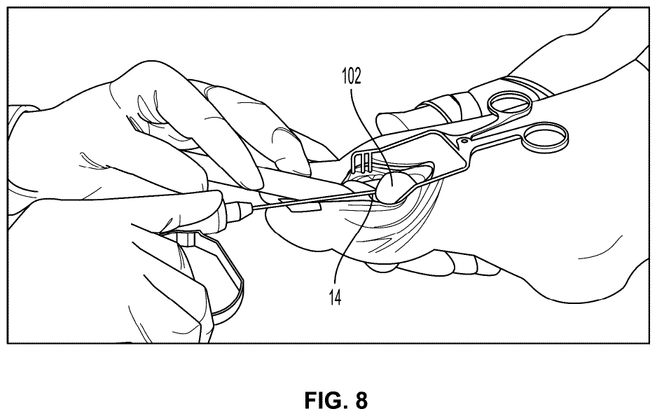

Instrument 10 can be used as a retractor to help gain access to the end of a bone for preparing the end face of the bone. For example, with reference to FIG. 8, head 14 of instrument 10 can be positioned under the plantar surface of the distal end of first metatarsal 102 (e.g., with the concave inner surface 22 facing the metatarsal bone). The clinician can manipulate the handle of the instrument to apply a plantar directed force to the handle, causing the head of the instrument to rotate dorsally and lift the distal end of first metatarsal 102 dorsally away from the proximal end of proximal phalanx 115. This can provide access for the clinician to insert a guidewire into the distal end of the first metatarsal and to ream the distal end of the bone. This also helps protect the proximal end of the proximal phalanx from potential damage due to the reamer. The clinician may additionally or alternatively perform a similar retraction and preparation of the proximal end of proximal phalanx 115.

After preparation of the ends of one or both bone ends, the clinician may realign the proximal phalanx relative to the metatarsal in one or more planes, such as the transverse plane and/or frontal plane and/or sagittal plane (e.g., each of the transverse, frontal, and sagittal planes). The clinician may or may not utilize a bone positioning device to facilitate movement of the bone portion. The moved position of the phalanx can result is realignment of the proximal end relative to the distal end of the metatarsal.

Independent of the type of tissue removing instrument used, the tissue removing instrument can be applied to the end face of the bone being prepared to remove cartilage and/or bone. For example, the tissue removing instrument may be applied to the end face to remove cartilage (e.g., all cartilage) down to subchondral bone. Additionally or alternatively, the tissue removing instrument may be applied to cut, fenestrate, morselize, and/or otherwise reshape the end face of the bone and/or form a bleeding bone face to promote fusion. In instances where a cutting operation is performed to remove an end portion of a bone, the cutting may be performed freehand or with the aid of a cutting guide having a guide surface positionable over the portion of bone to be cut. When using a bone preparation guide, a cutting instrument can be inserted against a guide surface (e.g., between a slot defined between two guide surfaces) of the bone preparation guide to guide the cutting instrument for bone removal.

After realignment (or in lieu of a separate realignment step), the clinician may or may not compress one or more bone faces of the opposed bone portions together (e.g., the end faces of the proximal phalanx and metatarsal). The clinician may compress the end faces together with hand pressure and/or using a compressing instrument physically attached to both the bones.

After suitably realigning at least a portion of the proximal phalanx relative to the first metatarsal, the clinician can fixate the position for fusion. In some examples, a provisional fixation step is performed in which one or more temporary fixation pins are deployed to hold the moved position of the phalanx (e.g., by inserting the fixation pin through the moved phalanx and into one or more adjacent bones). A permanent fixation device can be applied across the joint separating the prepared bone ends to hold a moved position of a bone for subsequent fusion. For example, one or more permanent fixation devices can be applied across the MTP joint. Example permanent fixation devices include, but are not limited to, pins (e.g., intramedullary nail, K-wire, Steinmann pin), plates, screws, staples, and combinations thereof. With time and healing, the realigned bone can subsequently fuse to the end face of the opposed bone to provide a fused joint.

In addition to use at the first metatarsophalangeal joint, instrument 10 and associated techniques of the disclosure may be used at other joints of the foot in which soft tissue release, bone mobilization, joint preparation, and/or protected access to a bone surface is useful. In such applications, head 14 may be selected, sized, and/or shaped based on the anatomy of the target joint while retaining the general configuration of a concave inner surface 22, a convex outer surface 24, and a sharpened leading edge 30. Thus, the clinician may place concave inner surface 22 against a selected bone end or articular surface and advance instrument 10 in a sweeping, reciprocating, and/or arcuate motion to cut capsule, ligament, tendon, fascia, scar tissue, and/or other soft tissue restraining the joint. Instrument 10 may additionally or alternatively be used as a retractor to distract adjacent bones, as a shield during bone preparation, and/or as a protector for adjacent cartilage, tendon, or other tissue while a saw, bur, osteotome, curette, rongeur, or reamer is used.

In some examples, instrument 10 is used at the talonavicular joint, which may also be referred to as the TN joint, between the talus and the navicular. For example, the clinician may surgically access the TN joint and position concave inner surface 22 of head 14 against a selected portion of the talar head and/or against an opposed articular region of the navicular. The sharpened leading edge 30 may then be advanced along medial, dorsal, plantar, and/or lateral aspects of the exposed bone end or joint space to release capsular and/or ligamentous tissue and thereby mobilize the talus relative to the navicular or the navicular relative to the talus. After release, instrument 10 may be maintained within or adjacent the joint to retract tissue, protect an opposed bone surface, and/or facilitate preparation of one or both joint surfaces for correction, realignment, arthrodesis, or other treatment.

In further examples, instrument 10 is used at the subtalar joint, which may also be referred to as the ST joint, between the calcaneus and the talus. In these applications, head 14 may be inserted into the subtalar joint space with concave inner surface 22 facing a selected articular portion of the talus and/or calcaneus. The clinician may manipulate handle 12 to move head 14 along one or more margins of the joint in order to release capsular tissue, ligamentous tissue, fibrous tissue, and/or other soft tissue that may restrict repositioning of the hindfoot. In some implementations, instrument 10 can help mobilize the talus relative to the calcaneus for deformity correction and/or subtalar fusion. Instrument 10 may also function as a retractor and shield while the clinician prepares one or both opposed surfaces of the talus and calcaneus and applies fixation to maintain a desired alignment.

In additional examples, instrument 10 and the disclosed techniques may be used at one or more lesser metatarsophalangeal joints, such as the second, third, fourth, and/or fifth MTP joints. For example, head 14 may be sized smaller than a head intended for the first MTP joint so that concave inner surface 22 more closely conforms to the distal end of a lesser metatarsal and/or the proximal end of an opposed proximal phalanx. The clinician may insert instrument 10 into the lesser MTP joint and use sharpened leading edge 30 to release collateral tissue, plantar soft tissue, dorsal capsular tissue, and/or other restraining tissue to mobilize the lesser toe for reduction, realignment, plantar plate-related correction, arthrodesis, or other treatment. As with the first MTP joint, instrument 10 may then be used to retract one bone relative to the other and/or to shield an opposed bone while bone end preparation and fixation are performed.

In still further examples, instrument 10 and associated techniques may be used at an interphalangeal joint of a lesser toe, such as a proximal interphalangeal joint, which may also be referred to as a PIP joint, for example during a hammer toe release procedure. In such applications, the clinician may access the contracted PIP joint and position head 14 against the proximal phalanx, the middle phalanx, and/or within the joint space. The sharpened leading edge 30 may be used to release contracted capsular tissue, collateral tissue, tendon-associated tissue, and/or other soft tissue contributing to the deformity. Following release, instrument 10 may facilitate distraction of the joint, protection of adjacent tissue, and access for resection, contouring, joint preparation, implant placement, and/or fixation associated with correction of the hammer toe deformity. Accordingly, although certain examples herein emphasize use at the first MTP joint, the disclosed instruments and techniques may be adapted for use at multiple forefoot, midfoot, and hindfoot joints without departing from the scope of the disclosure.

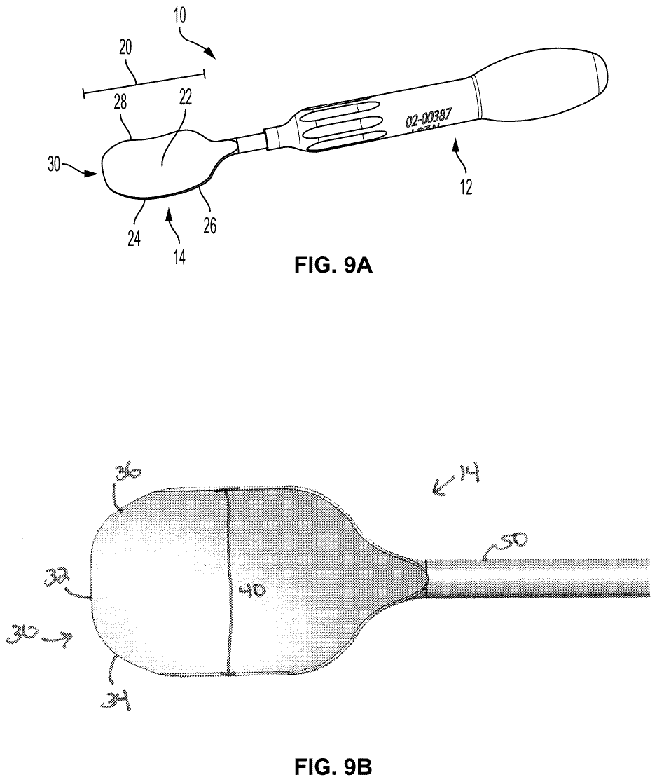

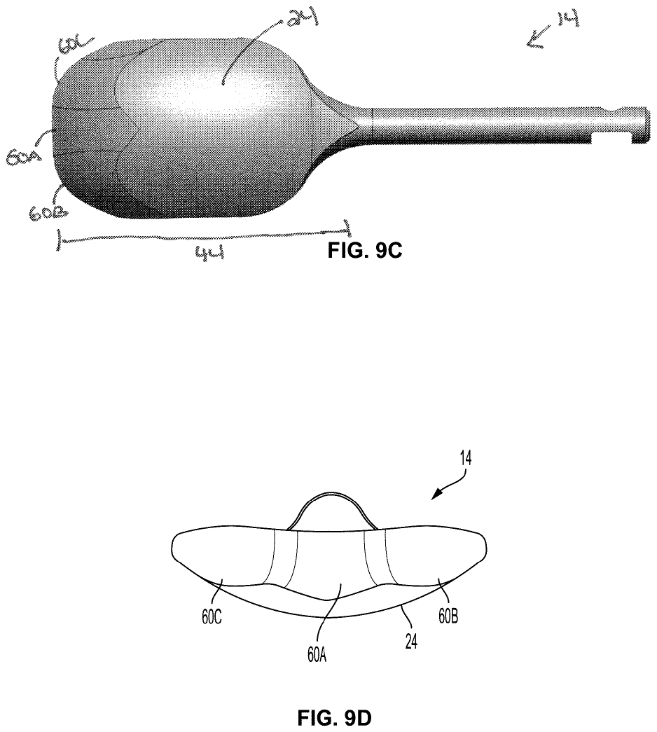

FIG. 9A-9D are different views of another example configuration of instrument 10 according to the disclosure. FIG. 9A-9D may correspond generally to the respective views shown in FIG. 1A-1D, but illustrate a different sizing arrangement of instrument 10. Like reference numerals in FIG. 9A-9D refer to like features previously described with respect to FIG. 1A-1D. In the illustrated example of FIG. 9A-9D, instrument 10 may have the same or similar general arrangement of handle 12 and head 14, but one or more relative dimensions of the instrument may differ from the example of FIG. 1A-1D. For example, head 14 in FIG. 9A-9D may have a comparatively greater width, such as a greater width across the leading region and/or across a trailing region of the head, while optionally maintaining a scooped configuration defined by concave inner surface 22 and convex outer surface 24.

In some applications, the comparatively wider sizing arrangement of FIGS. 9A-9D may be particularly useful when the instrument is used in a scooping manner around an end of a bone and/or along a joint surface to release soft tissue and mobilize a joint. For example, the increased width may provide increased coverage across a bone end, increased engagement with soft tissue adjacent the joint, and/or increased resistance to unintended over-advancement during a procedure. Such a configuration may be particularly suitable for use at midfoot and hindfoot joints, such as a talonavicular joint, a subtalar joint, a calcaneocuboid joint, a naviculocuneiform joint, an intercuneiform joint, and/or a tarsometatarsal joint, although the wider arrangement may also be used at other joints depending on the anatomy and procedural objective. In these and other applications, the scooped and comparatively wider head 14 may facilitate partial or full wrapping around an end of a first bone and/or a second bone to cut capsular tissue, ligamentous tissue, fibrous tissue, and/or other soft tissue adjacent the joint and thereby facilitate joint mobilization, bone preparation, realignment, fixation, fusion preparation, and/or other treatment steps.

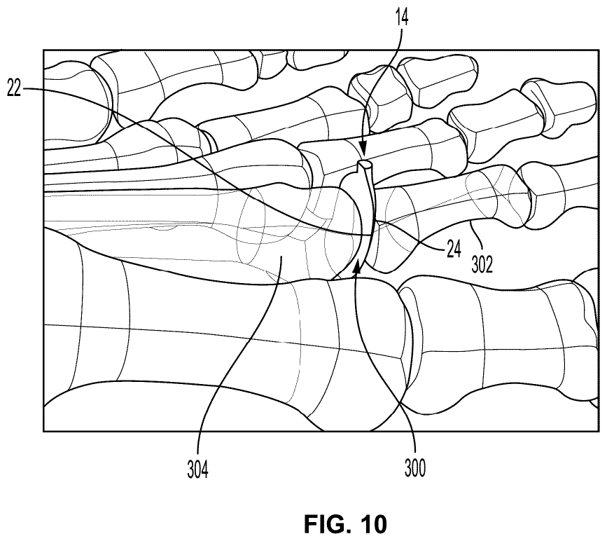

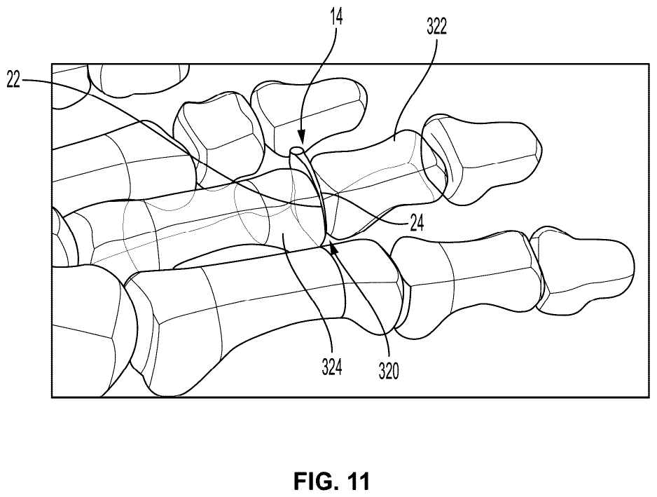

FIG. 10 is a perspective view of an example portion of a foot showing a lesser metatarsophalangeal joint 300 between a lesser proximal phalanx 302 and a lesser metatarsal 304. In the illustrated example, the lesser metatarsophalangeal joint 300 is the second metatarsophalangeal joint between the second metatarsal and the associated lesser proximal phalanx 302, although in other embodiments the disclosed techniques may be applied at another lesser metatarsophalangeal joint. As shown in FIG. 10, an example configuration of instrument 10 may be inserted into the lesser metatarsophalangeal joint 300 to release the joint. In the illustrated arrangement, the concave inner surface 22 may be positioned facing a distal end of the lesser metatarsal 304, with the convex outer surface 24 facing a proximal end of the lesser proximal phalanx 302.

In some examples, instrument 10 may be advanced, translated, rotated, and/or otherwise manipulated with the concave inner surface 22 facing the distal end of the lesser metatarsal 304 in order to cut, separate, and/or release capsular tissue, ligamentous tissue, fibrous tissue, and/or other soft tissue adjacent the lesser metatarsophalangeal joint 300. Additionally or alternatively, the orientation may be reversed such that the concave inner surface 22 faces the proximal end of the lesser proximal phalanx 302. In some techniques, the direction of insertion, advancement, and/or manipulation of instrument 10 may also be switched during the procedure, for example to facilitate release along different portions of the joint, to improve access to tissue adjacent one or both bones, and/or to achieve a desired degree of joint mobilization.

In some applications, after release of the lesser metatarsophalangeal joint 300, additional steps may be performed to prepare the joint for fusion between the lesser proximal phalanx 302 and the lesser metatarsal 304. For example, one or both opposed bone surfaces may be exposed and prepared by removing cartilage, resecting tissue, cutting, contouring, shaping, drilling, fenestrating, and/or otherwise preparing the distal end of the lesser metatarsal 304 and/or the proximal end of the lesser proximal phalanx 302 to promote arthrodesis. The lesser proximal phalanx 302 may then be repositioned relative to the lesser metatarsal 304 to place the toe in a desired alignment, orientation, and/or angular relationship. In some cases, fixation may then be applied to maintain the prepared bone surfaces in apposition during healing, such as with a pin, wire, screw, staple, plate, intramedullary implant, crossed fixation elements, and/or another fixation construct. Accordingly, the disclosed instrument 10 may be used as part of a lesser metatarsophalangeal fusion procedure in which joint release is followed by bone preparation, realignment, compression, fixation, and/or fusion.

FIG. 11 is a perspective view of an example portion of a foot showing a proximal interphalangeal joint 320 between a middle phalanx 322 and a proximal phalanx 324. In the illustrated example, the proximal interphalangeal joint 320 is the second proximal interphalangeal joint between the second proximal phalanx and the second middle phalanx, although in other embodiments the disclosed techniques may be applied at another proximal interphalangeal joint of a lesser toe. As shown in FIG. 11, an example configuration of instrument 10 may be inserted into the proximal interphalangeal joint 320 to release the joint. In the illustrated arrangement, the concave inner surface 22 may be positioned facing a distal end of the proximal phalanx 324, with the convex outer surface 24 facing a proximal end of the middle phalanx 322.

In some examples, instrument 10 may be advanced, translated, rotated, and/or otherwise manipulated with the concave inner surface 22 facing the distal end of the proximal phalanx 324 in order to cut, separate, and/or release capsular tissue, ligamentous tissue, fibrous tissue, tendon-associated tissue, and/or other soft tissue adjacent the proximal interphalangeal joint 320. Additionally or alternatively, the orientation may be reversed such that the concave inner surface 22 faces the proximal end of the middle phalanx 322. In some techniques, the direction of insertion, advancement, and/or manipulation of instrument 10 may also be switched during the procedure, for example to facilitate release along different portions of the joint, to improve access to tissue adjacent one or both bones, and/or to achieve a desired degree of joint mobilization.

In some applications, after release of the proximal interphalangeal joint 320, additional steps may be performed to prepare the joint for fusion between the proximal phalanx 324 and the middle phalanx 322. For example, one or both opposed bone surfaces may be exposed and prepared by removing cartilage, resecting tissue, cutting, contouring, shaping, drilling, fenestrating, and/or otherwise treating a distal end of the proximal phalanx 324 and/or a proximal end of the middle phalanx 322 to promote arthrodesis. The middle phalanx 322 may then be repositioned relative to the proximal phalanx 324 to place the toe in a desired alignment, orientation, and/or angular relationship. In some cases, fixation may then be applied to maintain the prepared bone surfaces in apposition during healing, such as with a pin, wire, screw, staple, intramedullary implant, and/or another fixation construct. Accordingly, instrument 10 may be used as part of a lesser toe proximal interphalangeal fusion procedure in which joint release is followed by bone preparation, realignment, fixation, and/or fusion.

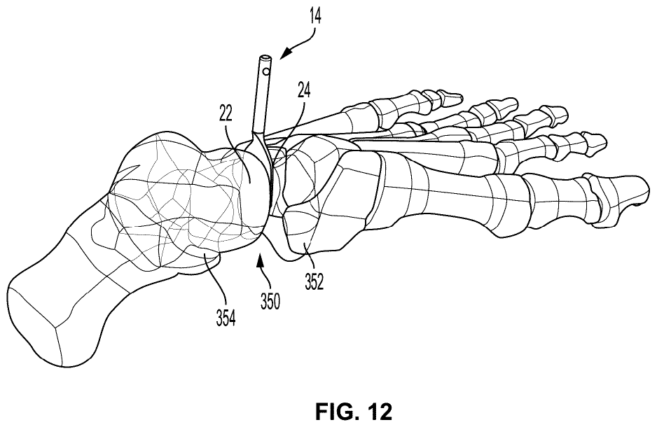

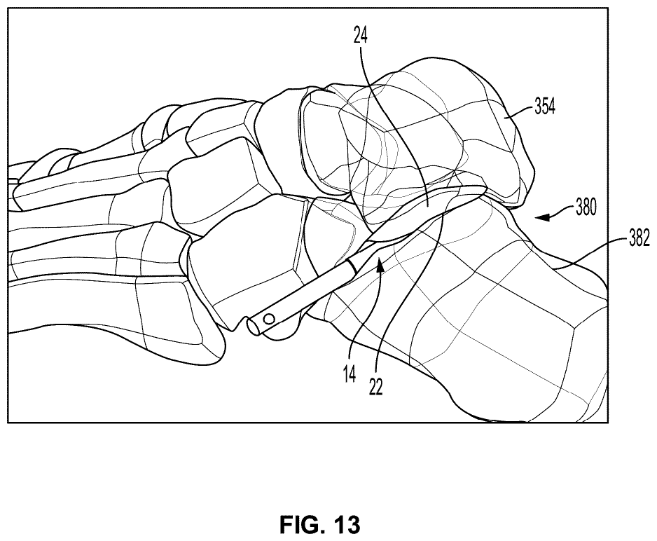

FIG. 12 is a perspective view of an example portion of a foot showing a talonavicular joint 350 between a navicular 352 and a talus 354. As shown in FIG. 12, an example configuration of instrument 10 may be inserted into the talonavicular joint 350 to release the joint. In the illustrated arrangement, the concave inner surface 22 may be positioned facing a distal end of the talus 354, with the convex outer surface 24 facing a proximal end of the navicular 352.

In some cases, instrument 10 may be advanced, translated, rotated, and/or otherwise manipulated with the concave inner surface 22 facing the distal end of the talus 354 in order to cut, separate, and/or release capsular tissue, ligamentous tissue, fibrous tissue, and/or other soft tissue adjacent the talonavicular joint 350. Additionally or alternatively, the orientation may be reversed such that the concave inner surface 22 faces the proximal end of the navicular 352. In some techniques, the direction of insertion, advancement, and/or manipulation of instrument 10 may also be switched during the procedure, for example to facilitate release along different portions of the joint, to improve access to tissue adjacent one or both bones, and/or to achieve a desired degree of joint mobilization.

In some applications, after release of the talonavicular joint 350, additional steps may be performed to prepare the joint for fusion between the navicular 352 and the talus 354. For example, one or both opposed bone surfaces may be exposed and prepared by removing cartilage, resecting tissue, cutting, contouring, shaping, drilling, fenestrating, and/or otherwise treating a proximal surface of the navicular 352 and/or a distal surface of the talus 354 to promote arthrodesis. The navicular 352 may then be repositioned relative to the talus 354 to place the bones in a desired alignment, orientation, and/or angular relationship. In some embodiments, fixation may then be applied to maintain the prepared bone surfaces in apposition during healing, such as with one or more screws, staples, plates, pins, wires, compression implants, and/or another fixation construct. Accordingly, instrument 10 may be used as part of a talonavicular fusion procedure in which joint release is followed by bone preparation, realignment, fixation, and/or fusion.

FIG. 13 is a perspective view of an example portion of a foot showing a subtalar joint 380 between a talus 354 and a calcaneus 382. As shown in FIG. 13, an example configuration of instrument 10 may be inserted into the subtalar joint 380 to release the joint. In the illustrated arrangement, the concave inner surface 22 may be positioned facing a distal end of the calcaneus 382, with the convex outer surface 24 facing a proximal end of the talus 354.

In some applications, instrument 10 may be advanced, translated, rotated, and/or otherwise manipulated with the concave inner surface 22 facing the distal end of the calcaneus 382 in order to cut, separate, and/or release capsular tissue, ligamentous tissue, fibrous tissue, and/or other soft tissue adjacent the subtalar joint 380. Additionally or alternatively, the orientation may be reversed such that the concave inner surface 22 faces the proximal end of the talus 354. In some techniques, the direction of insertion, advancement, and/or manipulation of instrument 10 may also be switched during the procedure, for example to facilitate release along different portions of the joint, to improve access to tissue adjacent one or both bones, and/or to achieve a desired degree of joint mobilization.

In some applications, after release of the subtalar joint 380, additional steps may be performed to prepare the joint for fusion between the talus 354 and the calcaneus 382. For example, one or both opposed bone surfaces may be exposed and prepared by removing cartilage, resecting tissue, cutting, contouring, shaping, drilling, fenestrating, and/or otherwise treating an opposing surface of the talus 354 and/or an opposing surface of the calcaneus 382 to promote arthrodesis. The talus 354 and calcaneus 382 may then be repositioned relative to one another to place the bones in a desired alignment, orientation, and/or angular relationship. In some examples, fixation may then be applied to maintain the prepared bone surfaces in apposition during healing, such as with one or more screws, staples, plates, pins, wires, compression implants, and/or another fixation construct. Accordingly, instrument 10 may be used as part of a subtalar fusion procedure in which joint release is followed by bone preparation, realignment, fixation, and/or fusion.

Various examples have been described. These and other examples are within the scope of the following claims.

1. A method of releasing soft tissue at a metatarsophalangeal joint, the method comprising:
surgically accessing a metatarsophalangeal joint between a first metatarsal and a proximal phalanx; and
moving a head of an instrument about a distal end of the first metatarsal, wherein the instrument comprises a concave inner surface, a convex outer surface opposite the concave inner surface, and a sharpened leading edge where the concave inner surface transitions to the convex outer surface;
wherein moving the head of the instrument about the distal end of the first metatarsal comprises positioning the concave inner surface facing the distal end of the first metatarsal and using the sharpened leading edge to cut soft tissue on a medial side, a lateral side, and a plantar side of the distal end of the first metatarsal.

2. The method of claim 1, wherein using the sharpened leading edge to cut soft tissue comprises cutting a medial collateral ligament and a lateral collateral ligament.

3. The method of claim 1, wherein using the sharpened leading edge to cut soft tissue comprises cutting sesamoid suspensory ligaments.

4. The method of claim 1, further comprising, after moving the head of the instrument about the distal end of the first metatarsal, reaming the distal end of the first metatarsal with a generally conical-shaped reamer.

5. The method of claim 1, wherein reaming the distal end of the first metatarsal comprises placing the concave inner surface of the head under the distal end of the first metatarsal and retracting the distal end of the first metatarsal relative to a proximal end of the proximal phalanx.

6. The method of claim 5, further comprising:
reaming a proximal end of the proximal phalanx with a generally cone-shaped reamer; and
applying fixation to fixate a position of the proximal end of the proximal phalanx relative to the distal end of the first metatarsal for fusion.

7. The method of claim 1, wherein the convex outer surface comprises a plurality of cutting faces extending from the leading edge proximally along at least a portion of a length of the convex outer surface.

8. The method of claim 7, wherein the plurality of cutting faces comprises three cutting faces positioned side-by-side from the first widthwise side edge to the second widthwise side edge.

9. The method of claim 1, wherein:
the concave inner surface defines a depth within a range from 6 mm to 10 mm; and
the concave inner surface defines a radius of curvature within a range from 10 mm to 20 mm.

10. The method of claim 1, wherein the leading edge of the head comprises:
a leading cutting surface;
a first side cutting surface extending angularly outwardly from the leading cutting surface to the first widthwise side edge; and
a second side cutting surface extending angularly outwardly from the leading cutting surface to the second widthwise side edge.

11. The method of claim 1, wherein
the head defines a maximum width;
the leading cutting surface has a length; and
the length of the leading cutting surface is less than the maximum width of the head.

12. The method of claim 1, wherein:
a width of the head is within a range from 15 mm to 20 mm; and
a length of the head over which the concave inner surface extends is within a range from 40 mm to 60 mm.

13. A method of releasing soft tissue at a joint of a foot, the method comprising:
surgically accessing a joint of a foot between a first bone and a second bone; and
moving a head of an instrument relative to the first bone and/or the second bone at the joint, wherein the instrument comprises a concave inner surface, a convex outer surface opposite the concave inner surface, and a sharpened leading edge where the concave inner surface transitions to the convex outer surface,
wherein moving the head of the instrument comprises positioning the concave inner surface facing an end of the first bone or an end of the second bone and using the sharpened leading edge to cut soft tissue adjacent the joint to mobilize the first bone relative to the second bone.

14. The method of claim 13, wherein the joint is one of a talonavicular joint between a talus and a navicular, a subtalar joint between a calcaneus and a talus, a lesser metatarsophalangeal joint, or a proximal interphalangeal joint of a lesser toe.

15. The method of claim 13, wherein using the sharpened leading edge to cut soft tissue comprises cutting capsular tissue and ligamentous tissue adjacent the joint.

16. The method of claim 13, further comprising, after moving the head of the instrument, preparing the end of the first bone and/or the end of the second bone for fusion.

17. The method of claim 13, wherein the convex outer surface comprises a plurality of cutting faces extending from the leading edge proximally along at least a portion of a length of the convex outer surface.

18. An instrument configured for cutting soft tissue at an end of a first bone or an end of a second bone defining a joint in a foot, the instrument comprising:
(a) a handle extending from a first end to a second end; and
(b) a head on the second end of the handle, wherein the head comprises:
(i) a concave inner surface extending from a first widthwise side edge of the head to a second widthwise side edge of the head and along at least a portion of a length of the head; and
(ii) a convex outer surface opposite the concave inner surface;
(iii) wherein the concave inner surface transitions to the convex outer surface along a leading edge, and the leading edge is sharpened along at least a portion of a length of the leading edge; and
wherein the head is sized to be inserted around the end of the first bone and/or around the end of second bone and configured to cut soft tissue for releasing the joint.

19. The instrument of claim 18, wherein:
the end of the first bone is a distal end of a first metatarsal; and
the head is sized to be inserted around the distal end of the first metatarsal with the concave inner surface facing the distal end of the first metatarsal, including plantarly of the distal end of a first metatarsal and in an intermetatarsal space between the first metatarsal and a second metatarsal of the foot.

20. The instrument of claim 18, wherein the joint is one of a talonavicular joint between a talus and a navicular, a subtalar joint between a calcaneus and a talus, a lesser metatarsophalangeal joint, or a proximal interphalangeal joint of a lesser toe.

21. The instrument of claim 18, wherein the convex outer surface comprises a plurality of cutting faces extending from the leading edge proximally along at least a portion of a length of the convex outer surface.

22. The instrument of claim 18, wherein:
the concave inner surface defines a depth within a range from 6 mm to 10 mm; and
a radius of curvature within a range from 10 mm to 20 mm.

23. The instrument of claim 18, wherein the leading edge of the head comprises:
a leading cutting surface;
a first side cutting surface extending angularly outwardly from the leading cutting surface to the first widthwise side edge; and
a second side cutting surface extending angularly outwardly from the leading cutting surface to the second widthwise side edge.

24. The instrument of claim 18, wherein:
the head further comprises a shank;
the handle is attached to the shank; and
a scooped body extends from the shank, the scooped body defined by the concave inner surface on one side and the convex outer surface on an opposite side.
